(19) United States
(12) Patent Application Publication
(10) Pub. No.: US 2018/0153476 A1
(43) Pub. Date: Jun. 7, 2018

(54) STROKE DETECTION USING BLOOD PRESSURE SURGE

(71) Applicant: Cardiac Pacemakers, Inc., St. Paul, MN (US)

(72) Inventors: Elizabeth Mary Annoni, White Bear Lake, MN (US); Pramodsingh Hirasingh Thakur, Woodbury, MN (US); Qi An, Blaine, MN (US); Sandra Nagale, Bolton, MA (US); Bryan Allen Clark, Forest Lake, MN (US); Thomas Christen, Needham, MA (US); Edward A. Goldberg, San Diego, CA (US)

(21) Appl. No.: 15/826,296

(22) Filed: Nov. 29, 2017

Related U.S. Application Data
(60) Provisional application No. 62/429,477, filed on Dec. 2, 2016.

(51) Int. Cl. A61B 5/00; A61B 5/0205; G06F 19/00

(52) U.S. Cl. CPC A61B 5/7275; A61B 5/0205; A61B 5/02125; G06F 19/3431; A61B 5/746

(57) ABSTRACT

This document discusses, among other things, systems and methods for detecting stroke. A system may comprise a sensor circuit for sensing a physiological signal, and a second sensor to detect a physical state change. The physical state change may include a transition in physical activity, posture, or sleep state. A stroke risk circuit may detect, from the sensed physiological signal, a signal indicative of blood pressure surge (BPS) in response to one or more physical state changes. The system may generate a stroke risk indicator indicating a risk of developing an impending stroke event using the detected BPS. The system includes an output unit that outputs the stroke risk indicator to a user or a process.

### CLAIM OF PRIORITY

This application claims the benefit of priority under 35 U.S.C. § 119(e) of U.S. Provisional Patent Application Ser. No. 62/429,477, filed on Dec. 2, 2016, which is herein incorporated by reference in its entirety.

### TECHNICAL FIELD

This document relates generally to medical devices, and more particularly, to systems, devices and methods for detecting stroke.

### BACKGROUND

Stroke is one of the leading causes of death and disability in the United States. A stroke may occur when the blood supply brain is interrupted or severely reduced, depriving the brain tissue of oxygen and nutrients. Within minutes, brain cells begin to die. About 85% of strokes are ischemic, characterized by blockages or narrowing of the arteries such as by blood clots, which may severely reduce the blood flow to the brain.

The faster a person with suspected stroke receives medical attention, the better their prognosis, and the less likely they will be to experience lasting damage or death. In order for a stroke patient to get the best diagnosis and treatment possible, they will need to be treated at a hospital within several hours of their symptoms first appearing. Treatment of stroke may depend on the type of stroke. For ischemic stroke, the treatment may include medications that dissolve blood clots and prevent further ones from forming, such as tissue plasminogen activator (tPA). Device therapy includes self-expandable stent retrievers that may be transvenously placed within the blocked or narrowed blood vessel to trap the clots.

### SUMMARY

Timely detection of an earlier indicator and diagnosis of a stroke is critical to reduce brain damage and death. However, prediction a stroke can be difficult. Usually there tends to be no pain associated with stroke. Patients may therefore miss the prime time for medical attention or the therapeutic window for medication administration. While the diagnosis of stroke may include blood test or imaging tests (e.g., CT scan, MRI scan, carotid ultrasound, or cerebral angiogram), the value of these tests are established provided that the patient can be timely transferred to the hospital. In an ambulatory setting when the patient is away from hospital, the diagnostic imaging may not be available for stroke prediction or for risk stratification.

Patient at risk of stroke may present with confusion, face drooping, arm weakness, trouble with speech, trouble with seeing, trouble with walking such as dizziness and lack of co-ordination, among other signs and symptoms. However, subjective description of these symptoms may be inaccurate and inconsistent. Ambulatory patients may not be able to communicate effectively the symptoms they experience upon a stroke. The information can also be biased due to a need for self-reporting or reliance on caregiver observations. For at least these reasons, the present inventors have recognized, among other things, substantial challenges and a demand for improved system and ambulatory devices to early detection or prevention of stroke.

Although stroke symptoms may appear at any time of the day or night, some patients may demonstrate a circadian pattern of arterial blood pressure with a peak incident in the morning (e.g., between 6 a.m. and noon) and the lowest incidence between midnight and 6 a.m. the next day. During the morning hours, patients at risk of stroke may experience a blood pressure surge (BPS) upon awakening, characterized by excessive increase in arterial blood pressure. The BPS may trigger strokes through a hemodynamic mechanism such as increased shear stress on the atherosclerotic cerebral vessels, increase of sympathetic nervous activity, platelet hyperactivity, hypercoagulability and hypofibrinolysis, blood viscosity, and increased vascular spasm. This document discusses, among other things, systems, devices, and methods for detecting stroke in a patient based at least on BPS. A system may comprise a sensor circuit for sensing a physiological signal, and a second sensor to detect a physical state change. The physical state change may include a transition in physical activity, posture, or sleep state. A stroke risk circuit may detect, from the sensed physiological signal, a BPS in response to one or more physical state changes, and generate a stroke risk indicator using the detected BPS. The system includes an output unit that outputs the stroke risk indicator to a user or a process.

Example 1 is a system for monitoring a patient at risk of a stroke. The system comprise a sensor circuit, a stroke risk circuit, and an output circuit. The sensor circuit may be coupled to a first sensor to sense a physiological signal indicative of a blood pressure surge (BPS), and a second sensor to detect a physical state change in the patient. The stroke risk circuit may be communicatively coupled to the first and second sensors, and configured to detect, from the sensed physiological signal, the BPS in response to the physical state change, and generate a stroke risk indicator using the detected BPS, the stroke risk indicator indicating a patient risk of stroke. The output unit configured to output the stroke risk indicator to a user or a process.

In Example 2, the subject matter of Example 1 optionally includes the second sensor that may include a posture sensor configured to detect a posture change. The stroke risk circuit may be configured to generate the stroke risk indicator using the BPS in response to the detected physical state change.

In Example 3, the subject matter of Example 2 optionally includes the posture sensor that may be configured to detect the posture change which may include: a transition from a lying-down position to sitting position; a transition from a sitting position to a standing position; or a transition from a lying-down position to a standing position.

In Example 4, the subject matter of any one or more of Examples 2-3 optionally includes the stroke risk circuit that may be configured to generate the stroke risk indicator using the sensed BPS in response to a posture change during a specified time of day.

In Example 5, the subject matter of Example 4 optionally includes the stroke risk circuit that may be configured to generate a stroke risk indicator using the sensed BPS in response to a posture change following a morning wakeup.

In Example 6, the subject matter of any one or more of Examples 1-5 optionally includes the second sensor that may include a sleep state detector configured to detect a sleep state transition from a first state to a second state. The stroke risk circuit may be configured to generate the stroke risk indicator using the sensed BPS in response to the detected sleep state transition.

In Example 7, the subject matter of Example 6 optionally includes the sleep state transition that may include a transition from a sleep state to an awakening state.

In Example 8, the subject matter of Example 6 optionally includes the sleep state transition that may include a transition between a rapid eye movement (REM) state and a non-REM state.

In Example 9, the subject matter of any one or more of Examples 1-8 optionally includes the first sensor that may be an ambulatory blood pressure sensor configured to sense the BPS including a change or a rate of change of a blood pressure in response to the detected physical state change.

In Example 10, the subject matter of any one or more of Examples 1-9 optionally includes the first sensor that may include a heart sound (HS) sensor configured to sense a HS component. The stroke risk circuit may be configured to generate the stroke risk indicator using a change in the sensed HS component in response to the detected physical state change.

In Example 11, the subject matter of any one or more of Examples 1-10 optionally includes the first sensor that may include a photoplethysmography (PPG) sensor configured to sense a pulse wave propagation parameter. The stroke risk circuit may be configured to generate the stroke risk indicator using a change in the sensed pulse wave propagation parameter in response to the detected physical state change.

In Example 12, the subject matter of any one or more of Examples 1-11 optionally includes the stroke risk circuit that may be configured to trend the BPS over time, and to generate the stroke risk indicator if the BPS trend exceeds a threshold.

In Example 13, the subject matter of any one or more of Examples 1-12 optionally includes the stroke risk circuit that may be configured to compute a statistical measure of the BPS trend, and to generate the stroke risk indicator if the BPS trend exceeds the threshold determined based on the statistical measure of the BPS trend.

In Example 14, the subject matter of any one or more of Examples 1-13 optionally includes an ambulatory medical device (AMID) that may include at least a portion of the stroke risk circuit and is communicatively coupled to the first and second sensors.

In Example 15, the subject matter of any one or more of Examples 1-14 optionally includes the output unit that may be configured to produce an alert to the user based on the stroke risk indicator.

Example 16 is a method for monitoring a patient at risk of a stroke. The method comprises steps of: sensing a physiological signal and a physical state change in the patient; detecting blood pressure surge (BPS) from the sensed physiological signal during physical state change; generating a stroke risk indicator using the detected BPS, the stroke risk indicator indicating a patient risk of stroke; and outputting the stroke risk indicator to a user or a process.

In Example 17, the subject matter of Example 16 optionally includes the step of sensing a physical state change which may include sensing a posture change; and detecting the BPS includes detecting the BPS during the detected posture change.

In Example 18, the subject matter of Example 17 optionally includes the posture change which may include: a transition from a lying-down position to sitting position; a transition from a sitting position to a standing position; or a transition from a lying-down position to a standing position.

In Example 19, the subject matter of any one or more of Examples 17-18 optionally includes the step of sensing the posture change that may include sensing the posture change during a specified time of day. \

In Example 20, the subject matter of any one or more of Examples 16-19 optionally includes the step of sensing a physical state change which may include detecting a sleep state transition from a first state to a second state; and detecting the BPS includes detecting the BPS during the detected sleep state transition.

In Example 21, the subject matter of any one or more of Examples 16-20 optionally includes the step of sensing the physiological signal indicative of BPS that may include sensing: a blood pressure; a heart sound (HS) component including one of a first (S1), second (S2), third (S3), or fourth (S4) heart sound; or a pulse wave propagation parameter including a pulse wave transit time or pulse wave velocity.

In Example 22, the subject matter of any one or more of Examples 16-21 optionally includes a step of trending the BPS over time. The generation of the stroke risk indicator may include comparing the BPS trend to a threshold.

In Example 23, a system may optionally combine any portion or combination of any portion of any one or more of Examples 1-22 to include “means for” performing any portion of any one or more of the functions or methods of Examples 1-22, or a “non-transitory machine-readable medium” including instructions that, when performed by a machine, cause the machine to perform any portion of any one or more of the functions or methods of Examples 1-22.

Detecting a patient risk of stroke using physiological sensors, such as discussed in this document, may improve medical diagnostics of stroke, as well as individualized therapies to improve patient outcome. The systems, devices, and methods discussed in this document may also enhance the performance and functionality of a stroke detection system or device. A device or a system programmed with the sensor-based stroke detection methods can have improved automaticity in medical diagnostics. More efficient device memory or communication bandwidth usage may be achieved by storing or transmitting medical information more relevant to clinical decisions. Additionally, through anti-stroke therapies based on patient individual need and therapy efficacy, battery longevity of an implantable device may be enhanced, or anti-stroke medication volume may be saved.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an example of a stroke monitoring system and portions of an environment in which the system may operate.

FIG. 2 illustrates an example of a stroke monitoring system.

FIG. 3 illustrates an example of a portion of a stroke monitoring system for detecting BPS during a specified physical state.

FIG. 4 illustrates an example of a method for detecting stroke in a patient.

FIG. 5 illustrates an example of a method for detecting stroke based on BPS.

FIG. 6 illustrates a block diagram of an example machine upon which any one or more of the techniques (e.g., methodologies) discussed herein may perform.

### DETAILED DESCRIPTION

Disclosed herein are systems, devices, and methods for detecting stroke. Physiological signal indicative of or correlated to blood pressure change may be sensed during one or more specified a physical state change, such as a transition in physical activity, posture, or sleep state. An indication of blood pressure surge (BPS) may be detected from the physiological signal. Based at least one the BPS, the system may generate a stroke risk indicator indicating a risk of developing an impending stroke event using the detected BPS. In other examples, the system can alert a clinician about the stroke detection, or alter or provide a therapy to treat an impending or detected stroke event or to prevent further damages caused by stroke.

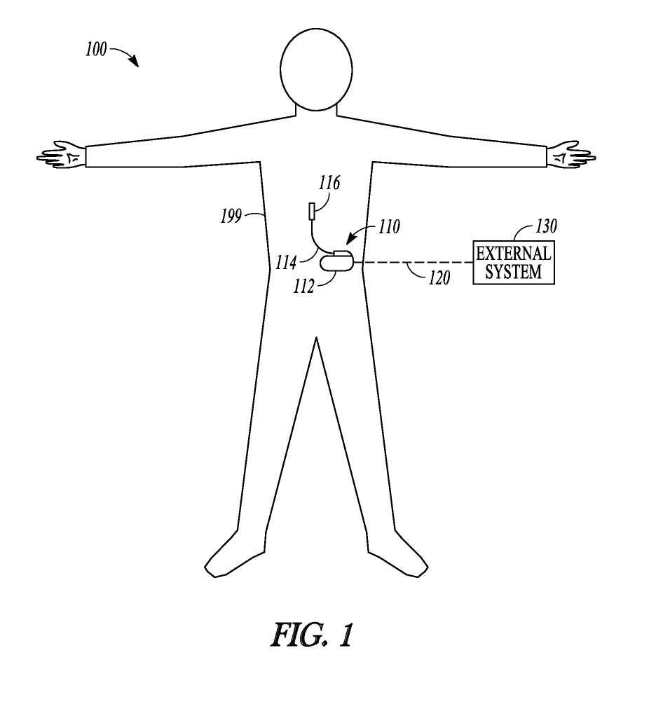

FIG. 1 illustrates, by way of example and not limitation, an example of a stroke monitoring system 100 and portions of an environment in which the system 100 may operate. The stroke monitoring system 100 may include a stroke monitor 110 that may be associated with a body of a patient 199, and an external system 130. A communication link 120 is provided by communication between the stroke monitor 110 and the external system 130.

The stroke monitor 110 may take the form of an ambulatory medical device (AMD) such as an implantable medical device (IMD) 112, a lead system 114, and one or more electrodes 116. The 1 MB 112 may be subcutaneously implanted in a chest, abdomen, or other parts of a patient 199. The 1 MB 112 may be configured as a monitoring and diagnostic device. The 1 MB 112 may sense physiological and functional signals in the patient, and predict an impending stroke (e.g., by detecting early indications or signs of stroke) or detect a stroke event. The 1 MB 112 may include a hermetically sealed can that houses a sensing circuitry, control circuitry, communication circuitry, and a battery, among other components.

The sensing circuitry of the 1 MB 112 may be configured to sense physiological or functional signals in the patient via sensing electrodes or ambulatory sensors associated with the patient. The physiological or functional signals may contain information about changes in a cardiovascular, hemodynamic, pulmonary, or neurological patient responses to physiological or functional changes that are correlated with, or contributing to, development of stroke symptoms. In some examples, the sensed physiological or functional signal may be indicative of or correlated to a blood pressure surge (BPS), such as detected during a change in patient physical state or at a particular time of the day. The 1 MB 112 may detect an indicator of BPS using the sensed physiological or functional signals. The 1 MB 112 may predict or detect a presence of stroke when the BPS satisfies a specified condition. The IMD 112 may generate an alert of the stroke or pre-stroke indication for the healthcare professionals, or to produce a recommendation for further diagnostic test or treatment.

In addition to patient monitoring and stroke detection, the IMD 112 may additionally include a therapy unit that may generate and deliver one or more therapies to the patient to prevent occurrence of stroke, or to treat or control stroke and complications to prevent further damage. The therapies may include electrical, magnetic, or other types of therapies. In some examples, the 1 MB 112 may include a drug delivery system such as a drug infusion pump for delivering medications to the patient, such as tissue plasminogen activator (tPA) that dissolves blood clots and thus restores or improves blood supply to the brain.

Although the discussion herein with respect to the stroke monitoring system 100 focuses on implantable system (such as the IMD 112), this is meant only by way of example and not limitation. It is within the contemplation of the inventors and within the scope of this document, that the systems, devices, and methods discussed herein may also be used implemented in, and executed by, a subcutaneous medical devices, wearable medical devices (e.g., watch-like devices, patch-based devices, or other accessories), or other ambulatory medical devices.

The external system 130 may be communicated with the 1 MB 112 via a communication link 120. The external system 130 may include a dedicated hardware/software system such as a programmer, a remote server-based patient management system, or alternatively a system defined predominantly by software running on a standard personal computer. The external system 130 may control the operation of the IMD 112, such as programming the IMD 112 for detecting stroke and optionally delivering therapies. The external system 130 may additionally receive via the communication link 120 information acquired by 1 MB 112, such as one or more physiological or functional signals. The external system 130 may include a display for displaying the physiological or functional signals, or alerts, alarms, emergency calls, or other forms of warnings to signal the detection of stroke.

In an example, the external system 130 may include an external data processor configured to analyze the physiological or functional signals received by the IMB 112, and to confirm or reject the detection of stroke. Computationally intensive algorithms, such as machine-learning algorithms, may be implemented in and executed by the external data processor, which may process the data retrospectively and provide an individualized prediction of an impending stroke such as to allow the patient to have enough time to react.

The communication link 120 may include one or more communication channels and intermediate devices between the external system and the IMD 112, such as a wired link, a telecommunication link such as an internet connection, or a wireless link such as one or more of an inductive telemetry link, a radio-frequency telemetry link. The communication link 120 may provide for data transmission between the 1 MB 112 and the external system 130. The transmitted data may include, for example, real-time physiological data acquired by the IMD 112, physiological data acquired by and stored in the IMD 112, therapy history data, data indicating device operational status of the IMD 112, one or more programming instructions to the IMD 112 which may include configurations for sensing physiologic signal or stimulation commands and stimulation parameters, or device self-diagnostic test, among others. In some examples, the 1 MB 112 may be coupled to the external system 130 further via an intermediate control device, such as a handheld external remote control device to remotely instruct the IMD 112 to generate electrical stimulation pulses in accordance with selected stimulation parameters produced by the external system 130.

Portions of the IMD 112 or the external system 130 may be implemented using hardware, software, firmware, or combinations thereof. Portions of the IMD 112 or the external system 130 may be implemented using an application-specific circuit that may be constructed or configured to perform one or more particular functions, or may be implemented using a general-purpose circuit that may be programmed or otherwise configured to perform one or more particular functions. Such a general-purpose circuit may include a microprocessor or a portion thereof, a microcontroller or a portion thereof, or a programmable logic circuit, or a portion thereof. For example, a “comparator” may include, among other things, an electronic circuit comparator that may be constructed to perform the specific function of a comparison between two signals or the comparator may be implemented as a portion of a general-purpose circuit that may be driven by a code instructing a portion of the general-purpose circuit to perform a comparison between the two signals.

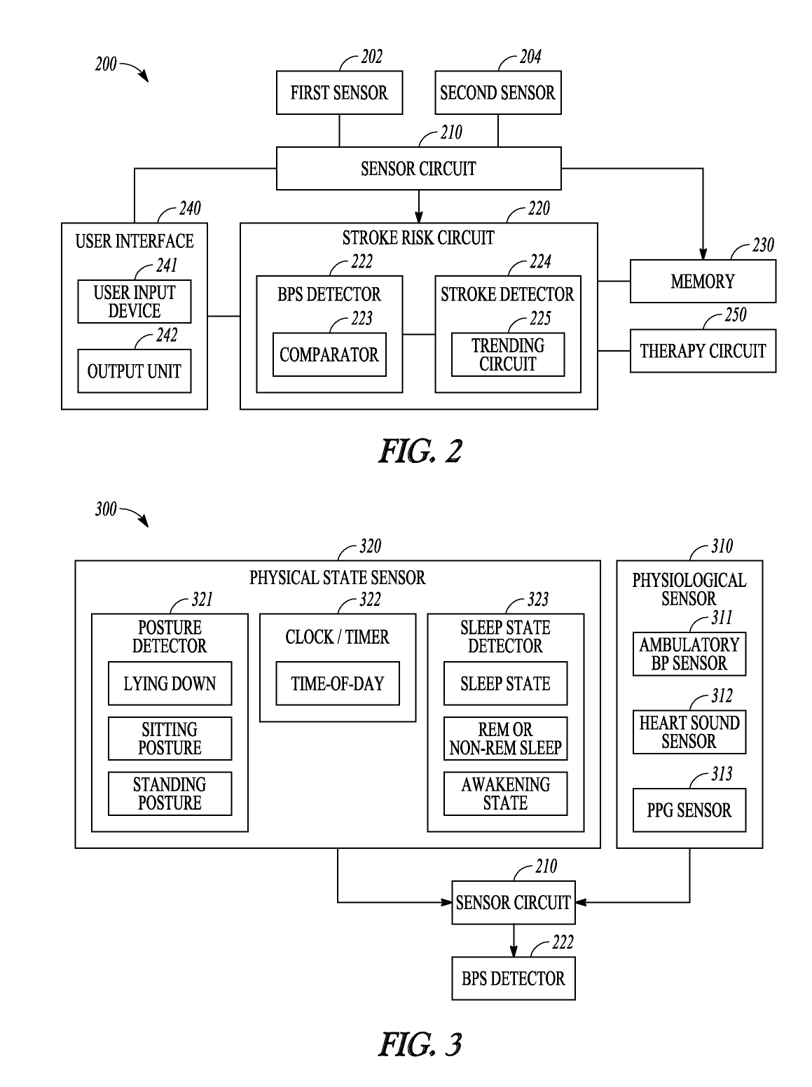

FIG. 2 illustrates generally an example of a stroke monitoring system 200, which can be an embodiment of the stroke monitoring system 100. The stroke monitoring system 200 may include a sensor circuit 210, a stroke risk circuit 220, a memory 230, and a user interface 240. The system 200 may optionally include a therapy circuit 250. In an example, at least a portion of one or more of the sensor circuit 210, the stroke risk circuit 220, the memory 230, the user interface 240, or the optional therapy circuit 250 may be included in an ambulatory device such as the 1 MB 112, or distributedly implemented between an ambulatory device and an external device such as a programmer or a remote patient management system.

The sensor circuit 210 may include sense amplifier coupled to a first sensor 202 and a second sensor 204. The first sensor 202 may sense a physiological signal, include cardiac, pulmonary, hemodynamic, neural, or biochemical signals. The physiological signal may contain information of blood pressure surge (BPS), or excessive increase in blood pressure. Examples of the physiological signal may include electrocardiograph (ECG), an electrogram (EGM), a heart rate signal, a heart rate variability signal, an intrathoracic impedance signal, an intracardiac impedance signal, an arterial blood pressure signal, a pulmonary artery pressure signal, a RV pressure signal, a LV coronary pressure signal, a blood pressure variability signal, a coronary blood temperature signal, a peripheral body temperature signal, a blood oxygen saturation signal, a heart sound (HS) signal, or a respiration signal (including, for example, respiration rate, tidal volume, minute ventilation, respiratory patterns), among others.

In an example, the sensor circuit 210 may be coupled to one or more electrodes such as on the lead system 114 and the can housing of the IMD 112, or one or more implantable, wearable, or other ambulatory sensors to sense the physiological or functional signals. Examples of physiological sensors may include pressure sensors, flow sensors, impedance sensors, accelerometers, microphone sensors, respiration sensors, temperature sensors, or blood chemical sensors, among others. In an example, the sensor circuit 210 may be coupled to a device capable of collecting or storing the physiologic information, such as an external programmer, an electronic medical record (EMR) system, or a memory unit, among other data storage devices.

The sense amplifier circuit can pre-process the physiological signals, including, for example, amplification, digitization, filtering, or other signal conditioning operations. The sensor circuit 210 may generate from the preprocessed physiological signal one or more signal metrics. The signal metrics may include temporal, morphological, or statistical features extracted from the physiological signal, and may be correlated to or indicative of variation in blood pressure.

The second sensor 204 may be configured to detect a physical state change in a patient, such as a transition from a first posture to a different second posture, or from a first physical activity level or a different second physical activity level. The physical state may be determined from a functional signal. In an example, the second sensor 204 may include an accelerometer configured to detect an activity intensity or activity duration. In another example, the second sensor 204 may include a tilt switch, an accelerometer, or a thoracic impedance sensor configured to detect posture or position. In various examples, the second sensor 204 may include gyroscope, magnetoresistive sensors, inclinometers, goniometers, electromagnetic tracking system (ETS), sensing fabric, force sensor, strain gauges, and sensors for electromyography (EMG) configured to sense motion, a gait, a balance, a locomotion pattern, a physical activity intensity or duration, among others. Examples of sensing the physical state change are discussed below, such as with reference to FIG. 3.

The stroke risk circuit 220 may include circuit sets comprising one or more other circuits or sub-circuits. The circuits or sub-circuits may, alone or in combination, perform the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

In various examples, the stroke risk circuit 220 may be implemented as a microprocessor circuit, such as a dedicated processor such as a digital signal processor, application specific integrated circuit (ASIC), microprocessor, or other type of processor for processing information including the physiological signals received from the sensor circuit 210. Alternatively, the microprocessor circuit may be a general purpose processor that may receive and execute a set of instructions of performing the functions, methods, or techniques described herein.

As illustrated in FIG. 2, the stroke risk circuit 220, which is communicatively coupled to the sensor circuit 210, may include a blood pressure surge (BPS) detector 222 and a stroke detector 224. The BPS detector 222 may detect from the sensed physiological signal an indication of BPS, which is represented by an excessive increase in blood pressure, when the patient undergoes a physical state change. The BPS detector 222 may calculate a change or a rate of change of a signal metric derived from the physiological signal measured during the detected physical state. A comparator 223 may compare the signal metric to a threshold (BPS_{TH}). If the signal metric satisfies a specified condition such as exceeding the threshold BPS TH by a specified margin, excessive blood pressure surge is deemed present. In an example, the threshold BPS TH is a rate of change of blood pressure, represented by an increase of ΔX millimeter of mercury (mmHg) in T hours. By way of example and not limitation, the threshold BPS_{TH} may be a pressure increase of approximately 20-50 mmHg in approximately 1-8 hours. In an example, the threshold BPS_{TH} is a blood pressure increase of 30 mmHg in 3 hours.

The threshold BPT_{TH} may be an individualized threshold that depends on the manner of physical state change. In an example, the threshold BPS_{TH} may be determined from an individualized baseline BPS. The baseline BPS for a particular patient may be calculated as a mean, a median, a mode, or other central tendency measure of prior measurements of the same signal metric during the same or similar physical state change (e.g., a specified posture change) over a specified time period (e.g., over past 5-30 days) when the patient is free of stroke event. Such an individualized baseline BPS takes into account physiological fluctuations in blood pressure or in a physiological parameter correlated to the blood pressure variation, such as due to circadian rhythm or reflective physiological changes. As such, the individualized baseline BPS may represent a patient stroke-free BPS level under the same or similar physical condition. If the BPS detector 222 detects a BPS level exceeding BPS_{TH} by a specified margin (e.g., approximately 10-20% of the baseline BPS level), then the surge in blood pressure is deemed excessive.

The stroke detector 224, coupled to the BPS detector 222, may generate a stroke risk indicator based on the detected BPS satisfying the specified condition. The stroke risk indicator indicates a patient risk of stroke. The stroke detector 224 may include a trending circuit 225 that may trend the BPS over time. A stroke risk indicator may be generated when the BPS trend exceeds a stroke detection threshold. In an example, the stroke detector 224 may use the BPS trend to generate a statistical measure, such as a histogram or an estimated statistical distribution of the trended BPS values. A stroke detection threshold may be determined from the histogram or the statistical distribution. The stroke detector 224 may detect the stroke, by generating the stroke risk indicator, if the BPS trend exceeds the stroke detection threshold. In an example, the stroke detection threshold may be determined as a percentile rank of the trended BPS values. A percentile rank, such as X-th percentile rank, refers to the BPS value where X % of the BPS values are equal to or less than that value. In an example, the BPS threshold may be chosen as 75-th percentile rank (BPS₇₅), such that 75% of the BPS values are less than or equal to BPS₇₅. The stroke risk indicator is generated when the BPS trend exceeds the threshold of BPS₇₅.

The memory 230 may be configured to store sensor signals or signal metrics such as generated by the sensor circuit 210, the BPS, and the stroke risk indicator. Data storage at the memory 230 may be continuous, periodic, or triggered by a user command or a specified event. In an example, a detection of BPS may trigger the data storage of the physiological signals. In an example, an interrogating device, such as a programmer in the external system 130 as illustrated in FIG. 1 and a remote server-based patient management system, may request access to the stored sensor signals, the BPS, and the stroke risk indicator stored in the memory 230. The requested information may be forwarded to the interrogating device such as via the communication link 120, where the information may be displayed or undergo further analysis, such as to confirm or reject the stroke detection.

The user interface 240 may include an input device 241 and an output unit 242. In an example, at least a portion of the user interface 240 may be implemented in the external system 130. The input device 241 may enable a user to provide parameters for sensing physiological or functional signals, parameters for detecting BPS, and stroke risk indicator. The input device 241 may include an input device such as a keyboard, on-screen keyboard, mouse, trackball, touchpad, touch-screen, or other pointing or navigating devices. The output unit 242 may generate a human-perceptible presentation of information including the detection of BPS and stroke risk indicator. The output unit 242 may include a display for displaying the information, or a printer for printing hard copies of the information. The information may be presented in a table, a chart, a diagram, or any other types of textual, tabular, or graphical presentation formats, for displaying to a system user. The presentation of the output information may include audio or other media format to inform the system user of the detected physiological events. In an example, the output unit 242 may generate alerts, alarms, emergency calls, or other forms of warnings to signal the system user about patient stroke risk.

The optional therapy circuit 250 may be configured to deliver a therapy to the patient in response to the detection of BPS and the risk of stroke. In an example, the therapy circuit 250 may control a drug infusion pump to deliver anti-stroke medication, such as tissue plasminogen activator (tPA). In another example, the therapy circuit 250 may deliver a rehabilitative therapy to treat or control side effects of stroke. The rehabilitative therapy may include electrostimulation therapy delivered to a neural target, or tissue or organs with impaired functions. In some examples, the anti-stroke therapy or rehabilitative therapy may be delivered in a closed-loop fashion. The therapy efficacy may be assessed based on sensor feedback. One or more therapy parameters may be adjusted, or drug dosage be tailored, based on the efficacy of the therapy delivered. In some examples, the therapy circuit 250 may provide assistive therapies to maintain adequate cardiorespiratory or hemodynamic support during and after a stroke. Examples of the assistive therapy may include respiratory rate regulation, heart rate regulation, cardiac pacing, or antiarrhythmic therapy, among others.

FIG. 3 illustrates generally an example of a portion 300 of a stroke monitoring system for detecting blood pressure surge (BPS) during a specified physical state. The system portion 300 may be an embodiment of a corresponding BPS detection portion of the stroke monitoring system 200 as illustrated in FIG. 2. The system portion may include a physiological sensor 310, a physical state sensor 320, a sensor circuit 210, and a BPS detector 222.

The physiological sensor 310, which may be an embodiment of the first sensor 202 of the stroke monitoring system 200, may include one or more sensors for measuring blood pressure or a physiological signal indicative of or correlated to blood pressure variation. One or more of these sensors may be implantable, wearable, or otherwise ambulatory. The physiological sensor 310 may include an ambulatory blood pressure sensor 311 configured to be positioned at or next to an artery or at a heart chamber, to invasively or noninvasively measure one of a peripheral arterial blood pressure (e.g., arterial pressure at finger, wrist, or arm), a pulmonary artery pressure signal, a RV pressure signal, a LV coronary pressure, a carotid artery pressure, among others.

The physiological sensor 310 may include sensors correlated to the changes in BP. By way of example and not limitation, one or more of a heart sound sensor 312, or a photoplethysmography (PPG) sensor 313 may be included, each of which may sense a signal that is correlated to, or contain information about, the BPS. The heart sound (HS) sensor 312 may include an accelerometer, an acoustic sensor, a microphone, a piezo-based sensor, or other vibrational or acoustic sensors may also be used to sense a HS signal. The HS sensor may be included in at least one part of an ambulatory system such as the 1 MB 112, or a lead coupled to the ambulatory medical device such as the lead system 114. The sensor circuit 210 may sense HS information including one or more HS components, such as first (S1), second (S2), third (S3), or fourth (S4) heart sound. The sensor circuit 210 may generate signal metrics from the HS signal, which may be correlated to or otherwise indicative of blood pressure surge. Examples of the HS signal metrics may include intensity of a HS component such as an amplitude or signal power of the S1 or S2 heart sounds, or HS-based cardiac timing interval such as pre-ejection period (PEP) such as measured between the onset of the QRS to the S1 heart sound, a systolic timing interval (STI) such as measured between the onset of the QRS complex on the ECG to the S2 heart sound, a left-ventricular ejection time (LVET) such as measured as an interval between 51 and S2 heart sounds, or a diastolic timing interval (DTI) such as measured between the S2 heart sound and the onset of the subsequent QRS complex on the ECG, among others.

The PPG sensor 313 may include pulse oximeter that illuminates the skin and measures changes in light absorption. The changes in light absorption may be correlated to the blood perfusion dynamics, which may reflect fluctuations in blood pressure. The PPG sensor may be position at a patient fingertip, ear, nasal septum, or forehead, among other locations. The sensor circuit 210 may generate from the PPG signal an indication of systolic, diastolic, or mean arterial pressure. In some examples, the sensor circuit 210 may generate one or more signal metrics indicative of pulse wave propagation, such as a pulse wave transit time elapsed from the first physiological event to the second physiological event, or a pulse wave velocity indicative of a propagation speed of the arterial pulse wave between the first and second physiological events.

The physical state sensor 320, which may be an embodiment of the second sensor 204 of the stroke monitoring system 200, may include one or more of a posture detector 321, a clock/timer 322, and a sleep state detector 323. The posture detector 321 may be coupled to a posture sensor, such as an accelerometer, a tilt switch, or a thoracic impedance sensor configured to detect a posture or position, which may include lying down, sitting, or standing postures. The posture detector 321 may detect a posture change, such as a transition from a lying-down position to sitting position, a transition from a sitting position to a standing position, or a transition from a lying-down position to a standing position. Such a posture change may cause blood pressure fluctuation. An excessive surge in BP during the posture change as discussed herein may be an indication of an elevated risk of stroke.

The clock/timer 322 may provide information about a time of day, such as a morning, or a particular time frame when the patient awakes during a day. Patients at risk of stroke may experience substantial blood pressure surge (BPS) in the morning, or at a time during the day upon wakeup, which may be contributed by an increase of sympathetic nervous activity and an elevated hemodynamic response.

The sleep state detector 323 may be coupled to a sensor to detect a sleep or awakening state, or a rapid-eye movement (REM) or non-REM sleep state. Examples of the sleep state sensors may include accelerometers, piezoelectric sensors, biopotential electrodes and sensors, or other physiologic sensors. These sensors may detect sleep states through brain activities such as via electroencephalograms (EEG), or systematic responses indicative of sleep states such as position, frequency of change of posture, intensity of activity, respiration, heart rate, or other physiological signal signals. The sensor circuit 210 may detect a transition from a sleep state to an awakening state. The sensor circuit 210 may additionally or alternatively detect a transition between a rapid eye movement (REM) sleep and a non-REM sleep. In patients with obstructive sleep apnea, it is identified that the blood pressure can surge during REM sleep particularly following obstructive respiratory events. In patients with hypertension, the blood pressure during REM may intermittently surge to values higher than during the daytime. BPS as occurred during a transition between the REM and non-REM sleep states may indicate an increased risk for strokes.

The BPS detector 222 may detect BPS using the signal metrics of the physiological signals sensed during a detected physical state transition. In an example, the BPS detector 222 may detect a “pre-awakening BPS” in response to a transition from sleep to awakening state, while the patient is still laying down on the bed prior to the postural transition to sitting or standing. The BPS may be measured as a change or a rate of change of a signal metric measured at the awakening state and that measured during sleep, such as when the blood pressure reaches the lowest level (indicated by the sensor signal metrics) during a specified period while sleeping. In another example, the BPS detector 222 may detect a “morning BPS” in response to a postural transition from lying down to standing as the patient awakes.

In some examples, the physiological signals may be sensed during a composite physical state change, such as sensed by one or more of the components or detectors of the physical state sensor 320. A composite physical state change is a combination of two or more of a posture change, a sleep state change, or time of day. For example, BPS may be measured during posture change from lying down to standing during a morning between 6 and 8 a.m., a posture change from lying down to standing following wakeup after a sleep or a nap, or a transition from sleep to awakening while the patient remains lying down on the bed prior to any posture change, etc.

In some examples, the BPS detector 222 may detect the BPS using multiple signal metrics from the physiological signals sensed during the physical state change. The BPS detector 222 may generate a composite BPS indicator using a linear or nonlinear combination of the signal metrics during the physical state change. Examples of the computation models may include a linear weighted combination, a nonlinear combination such as a decision tree, a neural network, a fuzzy-logic model, or a multivariate regression model, among others. In an example, the signal metrics may be respectively weighted by weight factors when they are combined. The weight factors indicate respective physiological signal reliability in evaluating the patient risk of developing a stroke. In an example, the reliability may be determined using historical data in the patient, including the physiological signals acquired during stroke in patient medical history. A signal metric that shows greater and more consistent changes in signal amplitude or signal power is deemed more reliable that another signal metric with smaller changes, or greater variability, in signal amplitude or signal power. A larger weight may be assigned to the more reliable signal metric than to a less reliable signal metric when establishing a linear or non-linear combination of the signal metrics. The comparator 223 may compare the composite BPS indicator to a predetermined condition such as a threshold to detect the presence of BPS. The detected BPS may be used by the stroke detector 224 to generate a stroke risk indicator.

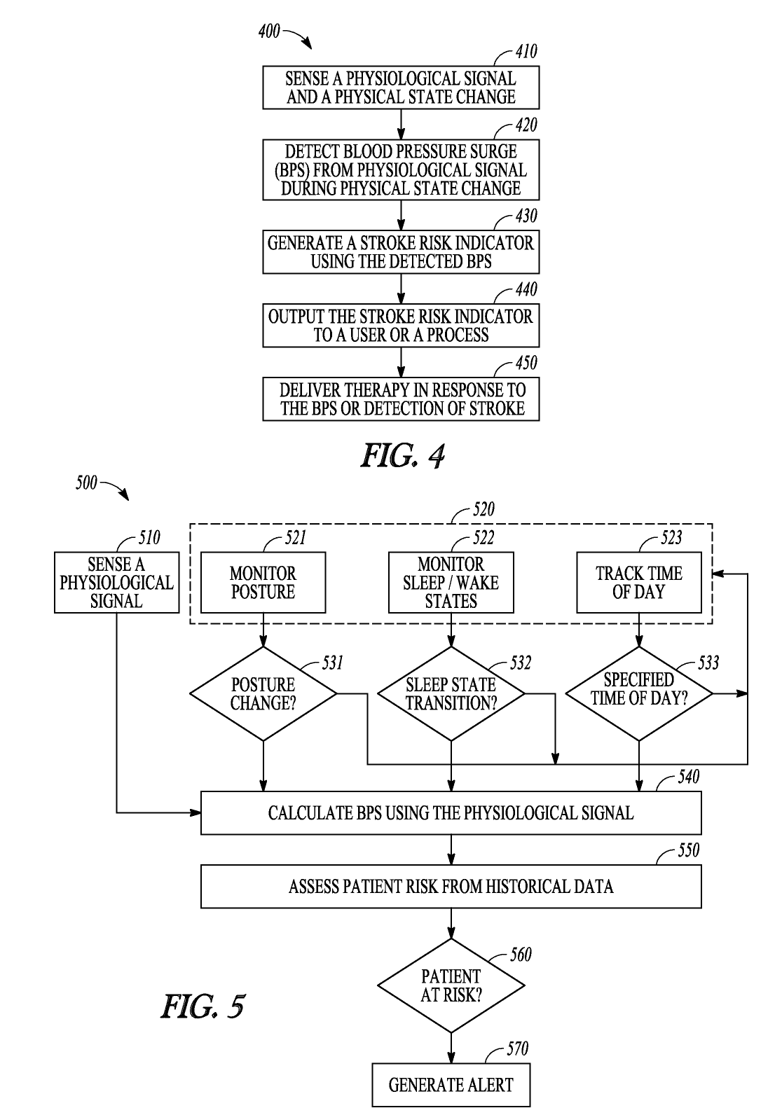

FIG. 4 illustrates generally an example of a method 400 for detecting stroke in a patient. The method 400 may be implemented and executed in an ambulatory medical device such as the IMD 112, or in a remote patient management system such as the external system 130. In an example, the method 500 may be implemented in and executed by the stroke monitoring system 200 in FIG. 2.

The method 400 begins at 410 by sensing a physiological signal and a physical state change in a patient. The physiological signal and the physical state may be sensed using respective sensors such as the first and second sensors 202 and 204 as discussed with reference to the stroke monitoring system 200 in FIG. 2. The physiological signal may be indicative of or correlated to blood pressure variation. Examples of the physiological signal may include electrocardiograph (ECG), an electrogram (EGM), a heart rate signal, a heart rate variability signal, an intrathoracic impedance signal, an intracardiac impedance signal, an arterial blood pressure signal, a pulmonary artery pressure signal, a RV pressure signal, a LV coronary pressure signal, a blood pressure variability signal, a coronary blood temperature signal, a peripheral body temperature signal, a blood oxygen saturation signal, a heart sound (HS) signal, or a respiration signal (including, for example, respiration rate, tidal volume, minute ventilation, respiratory patterns), among others. The physical state change may include a transition from a first posture to a different second posture, or from a first physical activity level or a different second physical activity level. The physical state may be determined from functional signals, such as a motion, a gait, a balance, a locomotion pattern, a physical activity intensity or duration, among others.

At 420, blood pressure surge (BPS) may be detected from the physiological signal during physical state change. A change or a rate of change of a signal metric of the physiological signal during the detected physical state change may be calculated. The change or rate of change of the signal metric may be correlated to or otherwise indicative of blood pressure change. Excessive BPS may be detected when the change in signal metric satisfies a specified condition, such as exceeding a BPS threshold representing a baseline BPS level during the same or similar physical state change when the patient is free of stroke event.

At 430, a stroke risk indicator may be generated using the detected BPS. The stroke risk indicator may be generated if a BPS trend exceeds a stroke detection threshold. The stroke detection threshold may be determined based on a statistical distribution of the BPS value, such as a BPS histogram. In an example, the stroke detection threshold may be chosen as X-th percentile rank computed from the BPS histogram.

At 440, the detection of the stroke may be output to a user or a process. In an example, a human-perceptible presentation of information, including the stroke risk indicator, may be generated and displayed such as on the output unit 242 of a user interface 240 as illustrated in FIG. 2. In an example, alerts, alarms, emergency calls, or other forms of warnings may be generated to signal an earlier detection of stroke.

The method 400 may optionally include a step 450 for delivering a therapy to the patient in response to the detection of BPS and the risk of stroke. The therapy may include drug therapy such as delivery of anti-stroke medications through a drug infusion pump device, and/or rehabilitative therapy to control side effects of stroke, such as electrostimulation therapy delivered to a neural target, or tissue or organs with impaired functions. The anti-stroke therapy or rehabilitative therapy may be delivered in a closed-loop fashion. In some examples, assistive therapies may be delivered at 450 to maintain adequate cardiorespiratory or hemodynamic support during and after a stroke.

FIG. 5 illustrates generally an example of a method 500 for detecting stroke based at least on the BPS. The method 500 may be an embodiment of the method 400, and may be implemented in and executed by the arrhythmia detection system 200 in FIG. 2, or the system portion 300 in FIG. 3.

The method 500 begins at 510 where a physiological signal may be sensed such as via an ambulatory physiological sensor. The physiological signal may include invasive or noninvasive blood pressure signal, such as a peripheral arterial blood pressure (e.g., arterial pressure at finger, wrist, or arm), a pulmonary artery pressure signal, a RV pressure signal, a LV coronary pressure, a carotid artery pressure. Alternatively or additionally, the physiological signals may include signals indicative of or correlated to blood pressure variation, which may include a heart sound signal, a photoplethysmography (PPG) signal, or an impedance signal.

One or more physical states including posture, sleep/awake state, or time of day may be monitored at 520, along with the physiological signal monitoring. Posture may be monitored at 521 such as using the posture detector 321. The posture change at 531 may include a transition from a lying-down position to sitting position, a transition from a sitting position to a standing position, or a transition from a lying-down position to a standing position. At 522, sleep or awakening state, or a rapid-eye movement (REM) or non-REM sleep state, may be detected such as using the sleep state detector 323. The sleep states may be detected using electroencephalograms (EEG), position, frequency of change of posture, intensity of activity, respiration, heart rate, or other physiological signal signals. Sleep state transition may be detected at 532, which may include a transition from sleep state to an awakening state, or a transition between REM sleep and a non-REM sleep. At 523, time of a day may be tracked, such as by using the clock/timer 322. A particular time frame, such as a morning between 6 a.m. and noon, or when the patient awakes during a day, may be identified at 533.

At 540, BPS may be calculated using the physiological signal detected during the physical state change. For example, a posture change may cause blood pressure fluctuation. If at 531 a specified posture change is detected, the physiological signal may be acquired to detect BPS. Similarly, if at 532 a sleep state transition such as from sleep to awakening, or between REM and non-REM sleep states, are detected, or if at 533 a specified time of a day is detected, the physiological signal may be acquired to detect BPS. In an example, a “pre-awakening BPS” in response to a transition from sleep to awakening state, while the patient is still laying down on the bed prior to the postural transition to sitting or standing. In another example, the BPS detector 222 may detect a “morning BPS” in response to a postural transition from lying down to standing as the patient awakes. In some examples, the physiological signal may be sensed when two or more of a posture change, a sleep state change, or time of day have been detected. For example, BPS may be measured during posture change from lying down to standing during a morning between 6 and 8 a.m., a posture change from lying down to standing following wakeup after a sleep or a nap, or a transition from sleep to awakening while the patient remains lying down on the bed prior to any posture change, etc.

In some examples, at 540 the BPS may be calculated using multiple physiological signals sensed during the physical state change. A composite BPS indicator may be generated using a linear or nonlinear combination of the signal metrics during the physical state change. Examples of the computation models may include a linear weighted combination, a nonlinear combination such as a decision tree, a neural network, a fuzzy-logic model, or a multivariate regression model, among others. In an example, the signal metrics may be respectively weighted by weight factors when they are combined. The composite BPS indicator may be compared to a predetermined condition such as a threshold to detect the presence of BPS.

At 550, a patient risk of stroke may be determined, such as based on patient medical record or disease history. The risk factors may include hypertension, cardiovascular diseases, diabetes, high cholesterol, obesity, smoking, alcohol use, or family history, among other medical and behavioral conditions. If at 560 the patient is recognized at an elevated risk, an alert may be generated at 570 to signal the healthcare provider a high likelihood of stroke evidenced by the BPS as detected at 540. In an example, recommendation for further diagnostic test or treatment may also be generated.

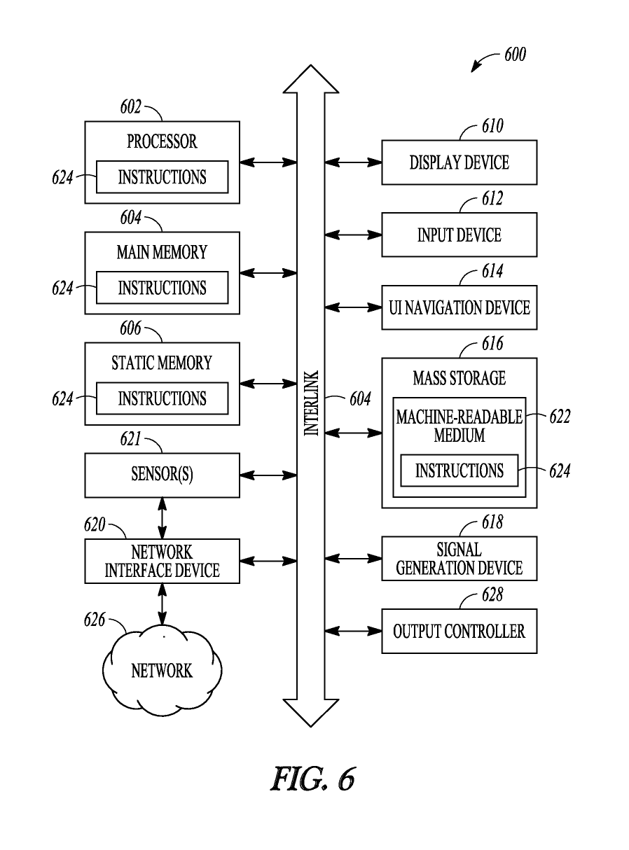

FIG. 6 illustrates generally a block diagram of an example machine 600 upon which any one or more of the techniques (e.g., methodologies) discussed herein may perform. Portions of this description may apply to the computing framework of various portions of the LCP device, the IMD, or the external programmer.

In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a display unit 610 (e.g., a raster display, vector display, holographic display, etc.), an alphanumeric input device 612 (e.g., a keyboard), and a user interface (UI) navigation device 614 (e.g., a mouse). In an example, the display unit 610, input device 612 and UI navigation device 614 may be a touch screen display. The machine 600 may additionally include a storage device (e.g., drive unit) 616, a signal generation device 618 (e.g., a speaker), a network interface device 620, and one or more sensors 621, such as a global positioning system (GPS) sensor, compass, accelerometer, or other sensor. The machine 600 may include an output controller 628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 616 may include a machine readable medium 622 on which is stored one or more sets of data structures or instructions 624 (e.g., software) embodying or utilized by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine readable media.

While the machine readable medium 622 is illustrated as a single medium, the term “machine readable medium” may include a single medium or multiple media (e.g., a centralized or distributed database, and/or associated caches and servers) configured to store the one or more instructions 624.

The term “machine readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine readable medium examples may include solid-state memories, and optical and magnetic media. In an example, a massed machine readable medium comprises a machine readable medium with a plurality of particles having invariant (e.g., rest) mass. Accordingly, massed machine-readable media are not transitory propagating signals. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 624 may further be transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software.

Various embodiments are illustrated in the figures above. One or more features from one or more of these embodiments may be combined to form other embodiments.

The method examples described herein can be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A system for monitoring a patient at risk of a stroke, the system comprising:
a sensor circuit, coupled to a first sensor to sense a physiological signal indicative of a blood pressure surge (BPS), and a second sensor to detect a physical state change in the patient;
a stroke risk circuit communicatively coupled to the first and second sensors, the stroke risk circuit configured to:
detect, from the sensed physiological signal, the BPS in response to the physical state change; and
generate a stroke risk indicator using the detected BPS, the stroke risk indicator indicating a patient risk of stroke; and
an output unit configured to output the stroke risk indicator to a user or a process.

2. The system of claim 1, wherein the second sensor includes a posture sensor configured to detect a posture change, and the stroke risk circuit is configured to generate the stroke risk indicator using the BPS in response to the detected physical state change.

3. The system of claim 2, wherein the posture sensor is configured to detect the posture change including:
a transition from a lying-down position to sitting position;
a transition from a sitting position to a standing position; or
a transition from a lying-down position to a standing position.

4. The system of claim 2, wherein the stroke risk circuit is configured to generate the stroke risk indicator using the sensed BPS in response to a posture change during a specified time of day.

5. The system of claim 1, wherein the second sensor includes a sleep state detector configured to detect a sleep state transition from a first state to a second state, wherein the stroke risk circuit is configured to generate the stroke risk indicator using the sensed BPS in response to the detected sleep state transition.

6. The system of claim 5, wherein the sleep state transition includes a transition from a sleep state to an awakening state.

7. The system of claim 5, wherein the sleep state transition includes a transition between a rapid eye movement (REM) state and a non-REM state.

8. The system of claim 1, wherein the first sensor is an ambulatory blood pressure sensor configured to sense the BPS including a change or a rate of change of a blood pressure in response to the detected physical state change.

9. The system of claim 1, wherein:
the first sensor includes a heart sound (HS) sensor configured to sense a HS component; and
the stroke risk circuit is configured to generate the stroke risk indicator using a change in the sensed HS component in response to the detected physical state change.

10. The system of claim 1, wherein:
the first sensor includes a photoplethysmography (PPG) sensor configured to sense a pulse wave propagation parameter; and
the stroke risk circuit is configured to generate the stroke risk indicator using a change in the sensed pulse wave propagation parameter in response to the detected physical state change.

11. The system of claim 1, wherein the stroke risk circuit is configured to trend the BPS over time, and to generate the stroke risk indicator if the BPS trend exceeds a threshold.

12. The system of claim 1, comprising an ambulatory medical device (AMID) that includes at least a portion of the stroke risk circuit and is communicatively coupled to the first and second sensors.

13. The system of claim 1, wherein the output unit is configured to produce an alert to the user based on the stroke risk indicator.

14. A method for monitoring a patient at risk of a stroke, the method comprising:
sensing a physiological signal and a physical state change in the patient;
detecting blood pressure surge (BPS) from the sensed physiological signal during physical state change;
generating a stroke risk indicator using the detected BPS, the stroke risk indicator indicating a patient risk of stroke; and
outputting the stroke risk indicator to a user or a process.

15. The method of claim 14, wherein:
sensing a physical state change includes sensing a posture change; and
detecting the BPS includes detecting the BPS during the detected posture change.

16. The method of claim 15, wherein the posture change includes:
a transition from a lying-down position to sitting position;
a transition from a sitting position to a standing position; or
a transition from a lying-down position to a standing position.

17. The method of claim 15, wherein sensing the posture change includes sensing the posture change during a specified time of day.

18. The method of claim 14, wherein:
sensing a physical state change includes detecting a sleep state transition from a first state to a second state; and
detecting the BPS includes detecting the BPS during the detected sleep state transition.

19. The system of claim 14, wherein sensing the physiological signal indicative of BPS includes sensing:
a blood pressure;
a heart sound (HS) component including one of a first (S1), second (S2), third (S3), or fourth (S4) heart sound; or
a pulse wave propagation parameter including a pulse wave transit time or pulse wave velocity.

20. The method of claim 14, further comprising trending the BPS over time, wherein generating the stroke risk indicator includes comparing the BPS trend to a threshold.
